(19) United States
(12) Patent Application Publication
(10) Pub. No.: US 2006/0158612 A1
(43) Pub. Date: Jul. 20, 2006

(54) Apparatus and method for topographical parameter measurements

(76) Inventors: Hans-Joachim Polland, Wolfratshausen (DE); Stefan Seitz ( Franzke), Germering (DE); Kristian Hohla, Vaterstetten (DE)

Correspondence Address: Bausch & Lomb Incorporated, One Bausch & Lomb Place, Rochester, NY, 14604-2701, US

(21) Appl. No.: 11/311,022

(22) Filed: Dec. 19, 2005

Related U.S. Application Data
(63) Continuation-in-part of application No. PCT/EP04/08196, filed on Jul. 22, 2004.

(30) Foreign Application Priority Data
Jul. 23, 2003 (DE) 10333558.7

(51) Int. Cl. A61B 3/14

(52) U.S. Cl. 351/206

(57) ABSTRACT

A topographical parameter measuring device and method utilizes a technique based on wave front reconstruction according to, e.g., Hartmann-Shack principles. The device includes a planar illuminator comprising a known array of illumination sources for projecting a light spot pattern onto a target surface. A CCD camera detects the positions of the reflected image spots in a manner similar to that in a Hartmann-Shack wave front sensor. The displacements of the light spots from reference coordinates are indicative of the slope of the surface at the plurality of sample points. A computational component is used to fit the slope data of a reference surface and the target surface to a polynomial, for example, a Zernike polynomial. The polynomial, properly weighted with the calculated coefficients, provides a continuous mapping of the elevation of the target surface. Based on the elevation data, all other topographical parameters including axial curvature, dioptric power, sphere, cylinder and others can be computed and displayed.

### RELATED APPLICATION DATA

This application is a Continuation-In-Part application of International Application No. PCT/EP2004/008196 filed on 22 Jul. 2004 and claims priority thereto under 35 USC 365(c) as well as to DE103 33 558.7 filed on 23 Jul. 2003.

### BACKGROUND

1. Field of the Invention

Embodiments of the invention are generally directed to apparatus and methods in the field of topography measurement; more particularly, in the field of diagnostic ophthalmology; and most particularly, directed to corneal topography.

2. Description of Related Art

Accurate topographical metrology provides valuable information for a variety of ophthalmic and non-ophthalmic (industrial) applications. Although the present disclosure will primarily refer to the measurement of physical and optical properties of an ophthalmic cornea, it is to be appreciated that the concepts and the apparatus and method embodiments of the invention described herein below are not so limited in application. They also apply to the topographical metrology of inorganic surfaces.

The physical and optical properties of the cornea that can be determined and/or derived from topographical measurements include corneal (anterior and/or posterior) surface curvature (e.g., radius of curvature typically expressed in millimeters; keratometry (K-readings), expressed in diopters); surface shape (e.g., measured as elevational variation from a reference surface, typically expressed in microns); corneal power (e.g., refraction, expressed in diopters); corneal structure and thickness (e.g., mechanical properties expressed in units of force, etc., and thickness measured in microns), and other properties. These categories can be further broken down. For instance, surface curvature (i.e., topography), the measure of the rate at which the surface bends at a particular location in a particular direction, may be expressed in the form of power maps in terms of axial (or sagittal) curvature and meridional (or tangential) curvature. Axial curvature is a measure of the curvature of a point on the surface in the axial direction relative to the center of the surface. Meridional curvature measures the curvature at a point on the surface in a meridional direction relative to other points at different diameter values. Elevation is a topographical parameter and is a different parameter than curvature (topography). The elevation value of a coordinate point on the corneal surface represents the height of the point relative to a reference surface. In ophthalmology, the reference surface may be a best-fit sphere determined in a proprietary manner by the instrument manufacturer. The difference between curvature and elevation is illustrated by an ablated section and an unablated section of a corneal surface. They both may have the same curvature, however, their relative heights with respect to a reference surface will be very different. Thus elevation data provides important information about ablation depth and optical zone size, for example, that curvature data cannot provide. Examples of applications assisted by these measurements include, without limitation, vision analysis, ophthalmic lens design and fitting, detection of corneal pathology, and diagnosis and photorefractive surgery.

Various types of corneal topography measuring devices are known, generally as keratoscopes, or more colloquially as topographers. A traditional type of topographer is a Placido-based device. In the Placido-type apparatus, an alternating bright/dark concentric ring pattern (Placido disk) is projected onto the anterior corneal surface. (Other patterns have been developed, such as checkerboard, spider web and others that operate on the same principles). A camera located along the optical axis detects the distorted reflected image of the Placido pattern. Directed image analysis performed by a computer provides certain topographical measurement parameters based on the deviation of the image from the undistorted Placido pattern. The results can then be displayed in various formats. It should be noted that Placido-based topographers cannot directly measure surface elevation because they do not record point coordinates (x,y,z) on the target surface. In addition, the central 1-1.5 mm region of the cornea cannot be examined due to the central aperture in the Placido disk to accommodate the on-axis camera.

Another type of topographer, developed by PAR Technology Corp., utilizes a rasterphotogrammetry-based technique. A fluorescein stain is topically applied to the corneal tear film, which is illuminated by a specific, fluorescing wavelength of light. The PAR (posterior apical radius) corneal topography system projects a known geometrical grid pattern onto the anterior corneal surface. Typically, two cameras view the surface from off-axis locations. The cameras image the distorted grid while the system employs a stereo-triangulation technique to directly measure topographic elevation data and compute axial and tangential curvatures and refractive power.

A third type of topographer uses slit scanning technology combined with Placido disk reflective imaging, as commercially embodied by the Orbscan brand corneal topography (more accurately, anterior segment analysis) system from Bausch & Lomb Incorporated. The Orbscan device can measure corneal thickness, surface elevation, anterior and posterior curvature parameters, anterior chamber depth and other parameters.

Other topographical measuring devices and methods are available that are based on ultrasonic imaging, confocal microscopy, optical coherence tomography and other optical interference techniques (e.g., Moiré). The limitations of these systems include technical complexity and high cost.

The elaborate algorithms used in modern keratoscopy-based metrology require that certain assumptions be made about the corneal shape, owing to the air-tear film interface of the refracting surface, or otherwise incorporate simplifications about the ocular structure to process their calculations. These assumptions and/or simplifications can adversely affect measurement accuracy, especially for unusual or irregular (e.g., keratoconic) comeas. Non-keratoscopy-based devices and techniques use more complex hardware and software to overcome the necessary corneal shape assumptions, however, their cost and complexity deter their commercial placement.

Wave front sensing and measurement of the eye's total optical aberrations is a complimentary ophthalmic technology developed over the past decade or so. Groundbreaking work in this field was described by Liang et al., Objective measurement of wave aberrations of the human eye with the use of a Hartmann-Shack wave-front sensor, J. Opt. Soc. Am. A 11, 7, pp. 1949-1957 (July 1994). Further technical advancements were disclosed by Liang and Williams in U.S. Pat. No. 5,777,719. Both of these references are hereby incorporated by reference in their entireties to the fullest allowable extent. In its basic form, the Hartmann-Shack wavefront sensor operates by illuminating the retina of the eye with a point source of semi-coherent light. The light reflected and scattered from the retina exits the eye over the full pupil diameter, having a wave front distorted by the lens and corneal components of the eye. A lenslet array images the reflected light in the pupil plane of the eye onto a detector in the form of an array of focal spots produced by the corresponding lenses of the lenslet array. A system computer computes the center of each focal spot (centroid) to specify its position coordinates (x′, y′) relative to the centroid positions (x, y) of a reference wave front image. The positional deviations (Δx, Δy) of the focal spot images describe the localized tilt or slope of the wave front at each sampled measurement location. As is well known, the partial derivatives of the measured wave front ∂(x′, y′) at each sampling position (x, y) are obtained from

$$\frac{\partial W ( {x}^{′} , {y}^{′} )}{\partial x} = Δ x / f , \frac{\partial W ( {x}^{′} , {y}^{′} )}{\partial y} = Δ y / f$$

where f is the focal length of the lenslets. Many approaches are known in the art for reconstructing the wave front from the calculated partial derivative values. One such popular approach is to use Zernike polynomials. The slope data are fitted with the sum of the first derivatives of a selected radial mode order of the Zernike polynomials using a least squares procedure to determine the values of the coefficients for each polynomial. Liang et al. (1994) limited his analysis to fourth-order Zernike polynomials, while Williams '719 utilized 65 Zernike polynomials representing a tenth-order analysis. The first-order Zernike modes represent the linear aberration terms. The second-order modes are the quadratic terms corresponding to the manifest refraction aberrations defocus and astigmatism. The third-order modes are the cubic terms that correspond to coma and coma-like aberrations. The fourth-order modes describe the main contribution from spherical aberration. The fifth to tenth-order modes represent local irregularities of the wavefront within the pupil. The weighted Zernike polynomials are then added together to obtain the total reconstructed wave aberration. It is noted that the above described calculations and their various manipulations underlie the physical operating principles of Hartmann-Shack wave front sensors and, as such, are well known in the art. See, e.g., MacRae et al., Customized Corneal Ablation, SLACK Incorporated, chapter six, p67 (2001) and the article entitled Wavefront interpretation with Zernike polynomials, Applied Optics 19 (9), pp 1510 (1980), both of which are hereby incorporated by reference in their entireties to the fullest allowable extent.

In view of the issues of measurement accuracy, device complexity, cost, measurement capability and other recognized challenges presented by commercially available topographers and their operating techniques, the inventors have recognised the need for device and method improvements. For example, it would be advantageous for a keratoscopy-based measurement to not require predictive assumptions about corneal shape or eye optics. In further view of the eloquent and versatile capabilities of ophthalmic wave front sensing technology, it would be beneficial to have less costly and less complex keratoscopy-based topographers that efficiently, accurately and directly measure and provide desired topographical parameters. These objects and others, along with their associated advantages, are realized in the embodiments of the invention described herein below and as set forth in the appended claims.

### SUMMARY OF THE INVENTION

Embodiments of the invention are generally directed to methods and apparatus used to measure topographical parameters of a surface. In its most general aspect, embodiments of the invention employ and/or implement a technique, for measuring a topographical parameter, that is generally known and used for making a wave front measurement with a Hartmann-Shack or other appropriate wave front sensor device. The known technique uses the displacement of light spot images to determine the slope values of the wavefront at sample point coordinates. The slope values are then fitted to the sum of the first derivatives of a Zernike polynomial to determine the polynomial coefficients. Once the desired polynomial is determined, a complete mapping of the reconstructed wave front is known. According to the embodiments of the instant invention, a similar analysis is applied to topographical surface measurement. The light spot images reflected from the corneal surface are detected and the image centroids are calculated to accurately determine the image positions relative to reference image positions. From the positions of the imaged spots it is possible to calculate the slope of the cornea at different sample locations. Once the slope data are known, it can be compared with similar data from a reference surface (e.g., reference sphere with a radius of 7.8 mm from which slope data would be expected for a regular cornea with a spherical shape). Thus the difference in the deviation of the slope data at the sample locations on the cornea can be calculated. Using the directly obtained derivative values of the surface coordinates, the known algorithm used for the calculation of the Zernike (or other polynomial form) polynomial for wave front reconstruction can be used to calculate and map the topographical parameters of the corneal surface. It is thus not necessary to develop a new algorithm for the calculation of the polynomial (Zernike or other) coefficients requiring additional verification and validation. The Zernike coefficients completely describe the elevation of the surface. Therefore, everything mathematically is known about the surface once the elevational data are obtained.

An embodiment of the invention is directed to a method for measuring a topographical parameter of a target surface. The method includes the steps of obtaining the positional coordinates of an image of a known array of illumination sources reflected from a reference surface at a known measurement location, obtaining the corresponding positional coordinates of an image of the known array of illumination sources reflected from a target surface at the known measurement location, and using a known Hartmann-Shack wavefront reconstruction procedure to determine a polynomial-based topographical representation of the target surface. According to an exemplary aspect, the representation of the target surface topography is expressed as a Zernike polynomial. Other polynomial representations can also be made including representation by, e.g., a Taylor Series, a Fourier Series, a Siedel polynomial, a bicubic spline, an orthogonal two-dimensional function and others known in the art.

According to a related embodiment, a method for measuring a topographical parameter of a target surface involves the steps of projecting light from a known plurality of light emitting sources onto a surface of the target, imaging a plurality of the projected light sources on the target surface onto a detector, determining a positional coordinate of each imaged light source on the detector, wherein each positional coordinate is determinative of a slope value of the target surface at each respective projected light source coordinate, determining a positional coordinate of each of a corresponding reference surface light source image on the detector, wherein each positional coordinate is determinative of a slope value of the reference surface at each respective projected light source coordinate, determining a difference between the slope of the target surface at each respective projected light source coordinate and the slope of the reference surface at each respective projected light source coordinate, wherein the slope difference represents a change in the deviation of the slopes of the target surface from the slopes of the reference surface, and determining the coefficients of a polynomial for the slope deviation values, wherein a continuous mapping of the curvature of the target surface is provided by the polynomial representation of the surface. In an aspect, the method further involves determining a relative elevational deviation value of the target surface at any surface coordinate location based upon the polynomial representation of the surface. The method for topographically mapping the target surface can be iterated to improve the topographical measurement accuracy. In an exemplary aspect, the target surface is the anterior surface of a human cornea. In an illustrative aspect, no more than two iterations are necessary to achieve the desired measurement accuracy. In a particular aspect, only one iteration is necessary. In various exemplary aspects, topographical maps, such as a curvature maps and an elevation map, can be constructed and displayed on a display device.

Another embodiment of the invention is directed to a topographical parameter measuring device. The device includes a measurement surface illuminator comprising a known array of illumination components arranged in a plane that is perpendicular to an axial measurement axis of the device, a distance measuring component for accurately measuring the distance between the illuminator and a measurement plane of the device, a camera and associated detector located along the axial measurement axis of the device in cooperative engagement with the illuminator and the distance measuring component, and a computational component programmed to calculate reference surface and target surface slope data from reflected reference and target surface illuminator image data and implement a Hartmann-Shack wavefront reconstruction algorithm to determine a polynomial-based topographical representation of the target surface. In an exemplary aspect, the Hartmann-Shack wavefront reconstruction algorithm utilizes an n^{th}-degree Zernike polynomial to represent the surface topography. In another exemplary aspect, the known array of illumination components of the illuminator consists of a plurality of LEDs in a defined pattern having known positions. In an aspect, the illuminator may include between 30 and 7500 LEDs. In a more particular aspect, the illuminator includes between 500 and 7500 LEDs. In an exemplary aspect, the illuminator includes between 30 and 300 LEDs. According to another aspect, the plurality of LEDs may collectively emit two or more colors of light for detection by a color sensitive camera. This color differentiation may provide for more reliable detection of reference data points and/or may provide data that can be distinguished more easily than that provided by mono-color illumination. In another illustrative aspect, the distance measuring component has a measuring accuracy equal to or better than 0.2 mm and, more particularly, equal to or better than 0.1 mm to obtain a refraction accuracy of 0.1 diopter. In various exemplary aspects, the distance measuring component can be a laser triangulation device, a slit lamp, an optical coherence tomography (OCT) device, an ultrasound device or other devices providing suitable measurement accuracy as known in the art.

In another embodiment, topographical parameter measurements as described herein could be made in an ‘online analysis’ manner. As used herein, the term ‘online analysis’ refers to the substantially simultaneous detection, measurement and display of topographical parameter information at a rate up to approximately 50 Hz and, illustratively, at a rate on the order of 25 Hz. In an illustrative aspect, 25 images of desired topographical parameters can be obtained online at a frequency of 25 Hz. The total measurement can thus be completed within one second. In a related aspect, movement of the eye could be determined over the measurement duration by simultaneously obtaining and tracking iris or pupil images of the eye.

These embodiments will now be described in detail with reference to the drawing figures.

### BRIEF DESCRIPTION OF THE DRAWINGS

FIG. 1 is a cross sectional block-type diagram of a topographical parameter measuring device according to an embodiment of the invention;

FIG. 2 is a diagram of an array pattern of illumination sources for a topographical parameter measuring device according to an exemplary embodiment of the invention;

FIG. 3 is a diagrammatic representation of the optical condition for detecting a reflected illumination beam according to an embodiment of the invention;

FIG. 4 is a photocopy of a camera image of a spherical glass reference surface and the image produced by an LED array configured as in FIG. 2 according to an embodiment of the invention;

FIG. 5 is a schematic drawing illustrating the physical geometric relationships used according to an embodiment of the invention;

FIG. 6 is a schematic diagram of a spherical reference surface to further assist the reader in understanding the relational parameters according to an embodiment of the invention;

FIG. 7 is a flow chart-type diagram depicting a measurement algorithm according to an embodiment of the invention;

FIGS. 8A, 8B are diagrammatic illustrations, respectively, of a target surface superimposed on a reference surface and the deviation of the curvature between the two surfaces according to an illustrative embodiment of the invention; and

FIG. 9 is a block-type process diagram illustrating further calculation steps according to a method embodiment of the invention.

### DETAILED DESCRIPTION OF A PREFERRED EMBODIMENT OF THE INVENTION

Embodiments of the invention are not limited to ophthalmic related apparatus and methods; however, since ophthalmic corneal topographical measurement is a principal application, the description of the various embodiments will exemplify ophthalmic applications.

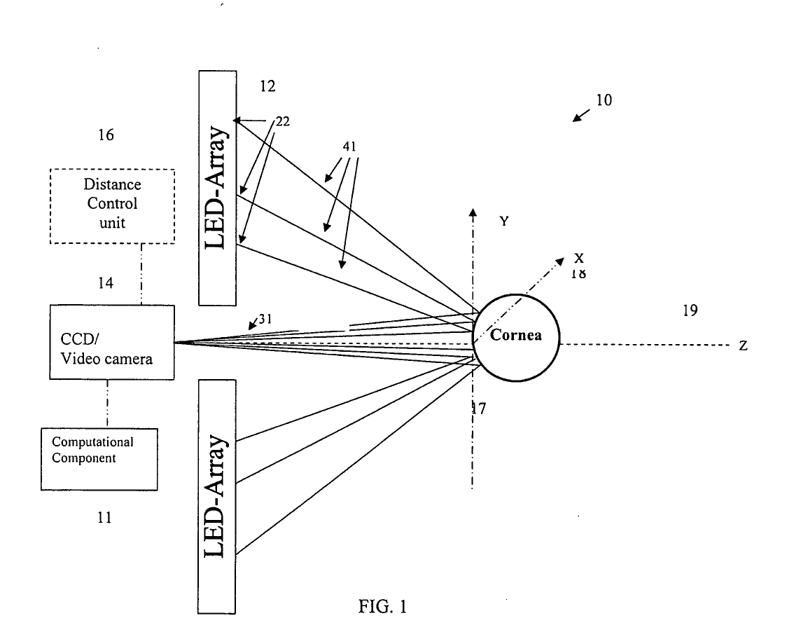

FIG. 1 schematically shows an exemplary ophthalmic topographical parameter measuring device 10. As used herein, the term ‘topographical parameter’ refers to any of a variety of known parameters including, but not limited to, surface curvature, corneal power, K-readings and elevation as these terms are understood in the art. As used herein, the term ‘topography’ of a surface will refer to the surface curvature; accordingly, elevation will be topographical data but will be considered distinct from topography (curvature). The device 10 includes a measurement surface illuminator 12 having a central paraxial aperture 31. The surface illuminator 12 lies in an X-Y plane that is perpendicular to a Z-coordinate axial measurement axis 19. A camera and associated detector 14 is located along the axial measurement axis 19 of the device on a posterior side of the illuminator. A distance measuring component 16 is in cooperative engagement with the illuminator 12 and the camera 14. The device 10 also includes a computational component 11 that is programmed to calculate reference surface and target surface slope data from reflected reference and target surface illuminator image data and implement a Hartmann-Shack wavefront reconstruction algorithm to determine a polynomial-based topographical representation of the target surface. Finally, the device has a measurement plane 17 where reference and target surfaces are to be located during measurement.

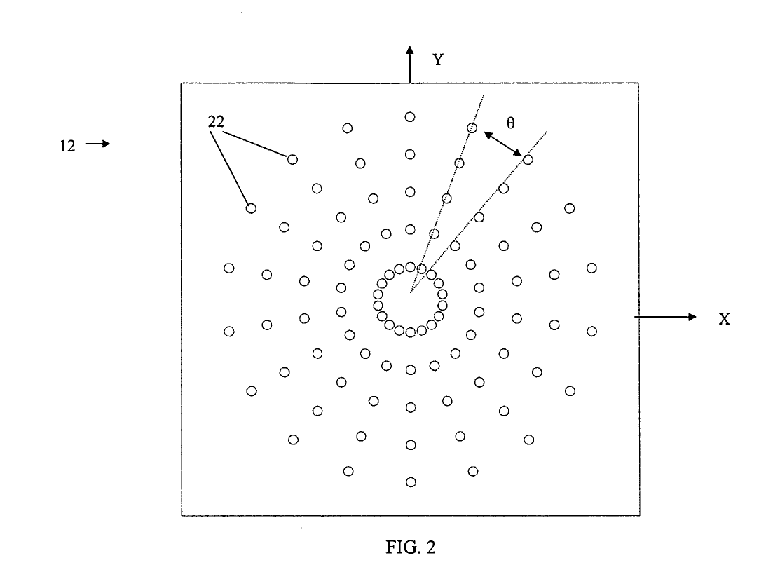

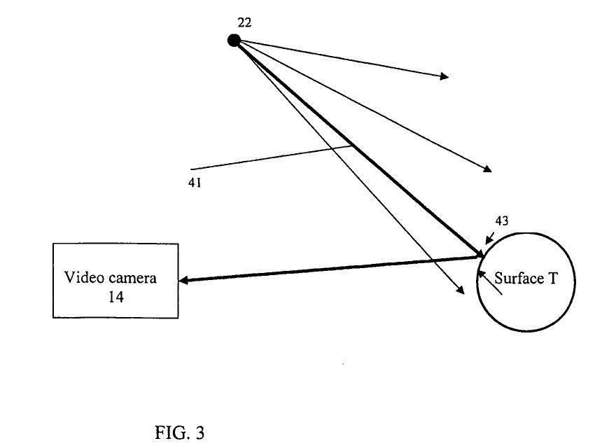

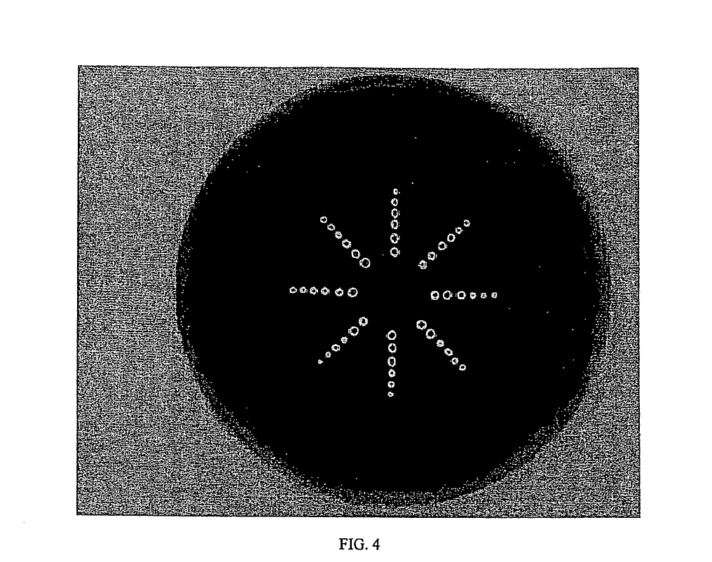

In an exemplary aspect illustrated with reference to FIG. 2, the illuminator 12 comprises a known array of LEDs 22; i.e., between about 30-300 LEDs 22 are arranged in a defined pattern having known positions with respect to the axial measurement axis 19 and other defined parameters as further described below. In an exemplary aspect, the configuration of the LED array is rotationally symmetric. As illustrated in FIG. 2, the LED array is in a pattern of five concentric rings or, alternatively, in a pattern of spoke-like straight lines emanating from a central hub. The positions of the LEDs 22 are advantageously chosen to obtain equidistant spots on the camera detector 14 for a spherical reference target T. In an illustrative aspect, the diameter of the reference target surface T is 7.8 mm. Although each of the LEDs 22 emits a fan of light, each LED can be considered to have a main (i.e., more intense) illumination beam path 41 that fulfils a reflection condition as illustrated in FIG. 3; that is, the highest intensity projection direction of the emitted light fan will be reflected by the surface 43 into the camera 14. As such, some of the LEDs, particularly those located farther from the center, may necessarily be tilted towards the vertex of the reference target or towards the pupil of the corneal target. FIG. 4 shows an actual camera image of the LED array pattern illustrated in FIG. 2 reflected from the spherical surface of a black glass ball.

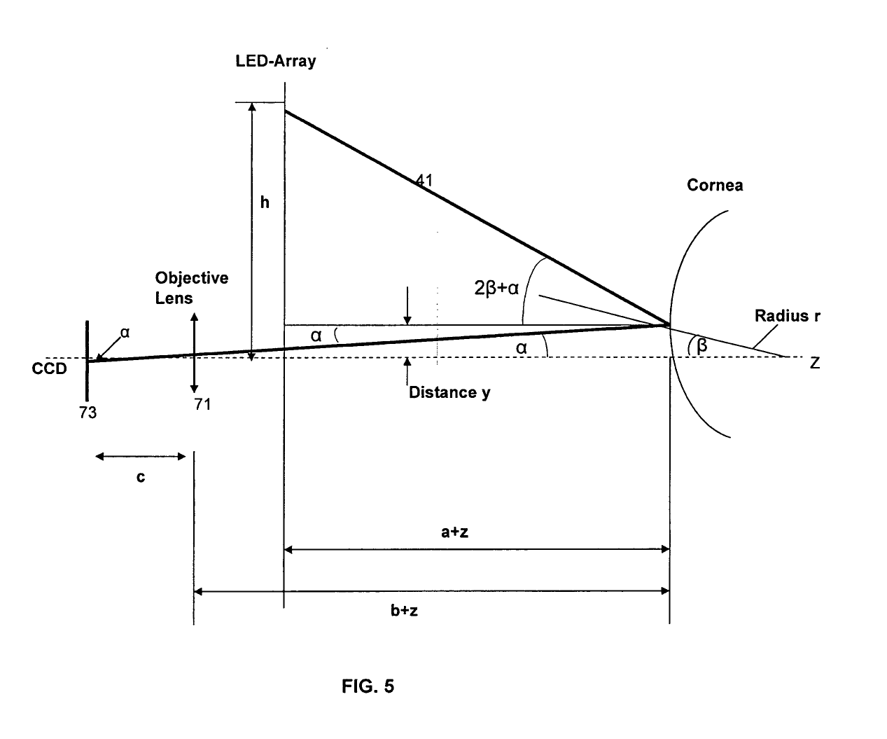

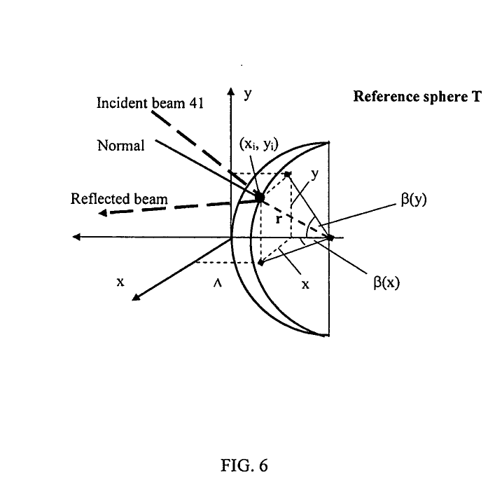

The design of the known LED array pattern can be determined from knowing the positions of the light spots in the camera image reflected from a reference surface. FIG. 5 illustrates the physical geometrical relationships for determining the design. With reference to the figure, ‘a’ is the distance (which can be varied by an amount z along the Z-axis) between the LED array plane 12 and the measurement plane 17 of the device 10. The measurement plane 17 is parallel to the LED array plane 12 and tangent to both the vertex of the reference surface T and the anterior corneal target surface T′. The distance ‘b’ (variable by an amount z along the Z-axis) is the distance between an objective lens 71 at the entrance of the CCD/camera 14 and the measurement plane 17. The distance between the center of the objective lens 71 and the CCD plane 73 is ‘c’. The value ‘r’ is the radius of a spherical reference target T. The vertical distance (height) of the surface illuminating beam 41 on the surface T from the optical measurement axis Z is defined as ‘y’. The illumination beam 41 reflects off of the surface T at an angle α as shown. Referring to FIG. 6, which shows the spherical reference surface T in greater detail, A is the distance along the Z-axis between the vertex of the surface and the vertical projection of a light spot at position (xᵢ, yᵢ) on the surface. The radial position of coordinate (xᵢ, yᵢ) is defined by β(x) and β(y) in x- and y-directions, respectively. The following relationships can be expressed for the calculation of β(x) and β(y):
x=(b+z+Δ)·tan(α(x))
y=(b+z+Δ)·tan(α(y))

$$tan ( α ( x ) + 2 \cdot β ( x ) ) = \frac{h - x}{a + z + Δ} ⇒ β ( x ) = 0.5 \cdot ( arctan ( \frac{h - x}{a + z + Δ} ) - α ( x ) )$$

$$tan ( α ( y ) + 2 \cdot β ( y ) ) = \frac{h - y}{a + z + Δ} ⇒ β ( y ) = 0.5 \cdot ( arctan ( \frac{h - y}{a + z + Δ} ) - α ( y ) )$$

For a sphere with radius r the following formula can be used for the calculation of x and y and Δ:

$$x = \frac{r \cdot tan ( β ( x ) )}{\sqrt{1 + {tan}^{2} ( β ( x ) ) + {tan}^{2} ( β ( y ) )}}$$

$$y = \frac{r \cdot tan ( β ( y ) )}{\sqrt{1 + {tan}^{2} ( β ( x ) ) + {tan}^{2} ( β ( y ) )}}$$

$$Δ = r - \sqrt{{r}^{2} - {x}^{2} - {y}^{2}}$$

The following exemplary parameter values were used to calculate the vertical height values, h, shown in Table 1, of the LED-positions in the array plane in relation to the corresponding ‘y’ values (for x=0):

| | |
|---|---|
| a: | 60.0 mm |
| b: | 80.0 mm |
| z: | 0.00 mm |
| a + z: | 60.0 mm |
| b + z: | 80.0 mm |
| Radius r | 7.80 mm |

TABLE I

| y | β | Δ | α | α + 2*β | h | |
|---|---|---|---|---|---|---|
| 0.0 mm | 0.00° | 0.00 mm | 0.00° | 0.00° | 0.00 | mm |
| 0.5 mm | 3.68° | 0.02 mm | 0.36° | 4.03° | 8.62 | mm |
| 1.0 mm | 7.37° | 0.06 mm | 0.72° | 8.08° | 17.60 | mm |
| 1.5 mm | 11.09° | 0.15 mm | 1.07° | 12.16° | 27.34 | mm |
| 2.0 mm | 14.86° | 0.26 mm | 1.43° | 16.28° | 38.41 | mm |
| 2.5 mm | 18.69° | 0.41 mm | 1.78° | 20.47° | 51.72 | mm |
| 3.0 mm | 22.62° | 0.60 mm | 2.13° | 24.75° | 68.84 | mm |
| 3.5 mm | 26.66° | 0.83 mm | 2.48° | 29.14° | 93.02 | mm |
| 4.0 mm | 30.85° | 1.10 mm | 2.82° | 33.68° | 132.26 | mm |
| 4.5 mm | 35.23° | 1.43 mm | 3.16° | 38.40° | 213.65 | mm |

Alternatively, the angle β values can be determined if α, h, a and b are known. The values of a and b can be determined by a calibration procedure and distance measurement as described below. The distance h of the LED from the central measurement axis is known from the design, and the angle α is measured with the video camera.

In an alternative aspect of the device, at least one, or more, of the LEDs 22 in the array 12 could emit at least one different color of light than other LEDs. In this case, the camera 14 will be a color-sensitive camera. The one or more colored LEDs may help to distinguish between the LED images in a more secure way. For example, it could be helpful to use one single LED with another color to serve as a reference point; or, a particular sub-pattern of LEDs could be made with another color in the illuminator, which may also be helpful as a reference pattern. In another aspect, an illumination source controller (not shown) could operably be connected with the device 10 to provide selective control of the plurality of illumination components.

As illustrated in FIG. 1, a distance measuring component 16 is provided for accurately measuring the distance (a+z) between the illuminator plane 12 and the measurement plane 17. In the exemplary embodiment, the measurement plane of the corneal target surface T′ is about 60 mm from the illuminator plane. Approximate control of the distance can be obtained with the aid of an adjustable objective lens 71 at the camera entrance. The image of the iris of the subject's pupil can be focused, noting that the iris is located approximately 3 mm posteriorly to the apex of the cornea. Precise distance control to within 0.2 mm and, more particularly, to within 0.1 mm accuracy can be achieved with the distance measurement component 16. It is suggested that this level of accuracy be maintained for precise topographical measurements. A laser triangulation device, an ophthalmic slit lamp, an OCT device and an ultrasonic device are examples of appropriate distance measurement components, as well as others known in the art. In a particular aspect, reference spheres with known radius values accurate to 0.1% are used to calibrate the particular distance measurement component used in the system 10.

As indicated above, the device 10 also includes a computational component 11. The computational component is programmed to execute an algorithm, described in detail below, to calculate reference surface (T) and corneal target surface (T′) slope data from reflected reference and target surface illuminator image data and implement a Hartmann-Shack wavefront reconstruction algorithm to determine a polynomial-based topographical representation of the target surface. Thus a programmable instruction or a reference for accessing a programmable instruction for carrying out the measurement algorithm may be a resident component of the computational component. Alternatively, the computational component may be equipped to accept a readable medium containing the algorithm instruction or reference.

In an exemplary operational set-up as illustrated in FIG. 1, the corneal surface T′ of the subject's eye 18 is located at the measurement plane 17 of the device along the central measurement axis 19 at a known distance from a video camera/CCD detector 14. The LED array plane forming the illuminator 12 has an aperture 31 around its center point through which passes the central measurement axis 19. The camera 14 is thus paraxially located with respect to measurement axis 19. Light projected from the illumination sources 22 towards the corneal surface T′ is reflected into the camera/detector 14 as illustrated.

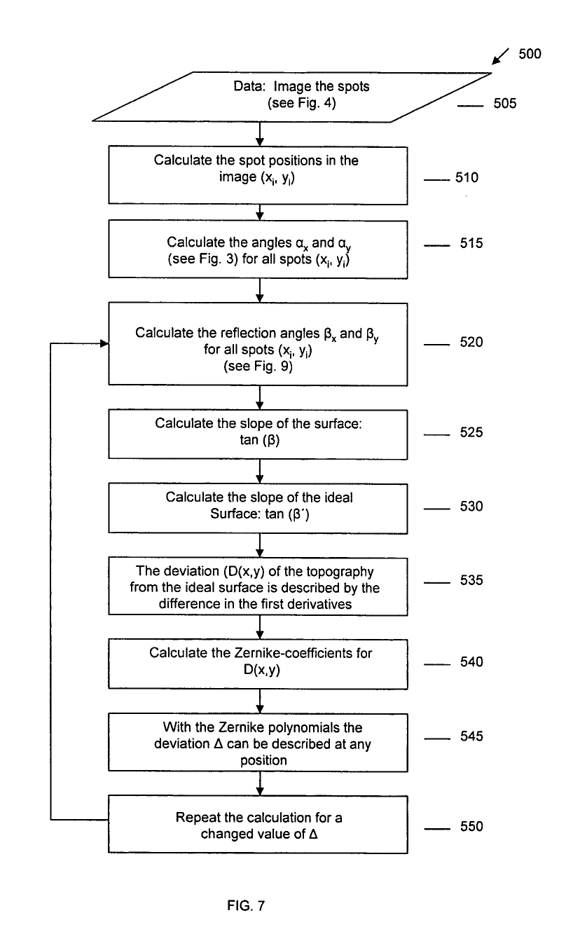

Another embodiment of the invention is directed to a method for measuring a topographical parameter of a target surface T′. The flow chart 500 in FIG. 7 sets forth the process steps with reference to FIGS. 1, 5 and 6. At step 505 a plurality of the light spots on the target surface (T′(xᵢ,yᵢ)) created by the known plurality of light sources 22 (L(xᵢ,yᵢ)) are imaged onto a CCD detector 73 of camera 14. At step 510 the positional coordinates (P′(xᵢ,yᵢ)) of each imaged light spot are calculated. Known calculation methods utilizing centroid finding algorithms, for example, can be used to determine the image coordinates. At step 515 the reflection angle components αₓ, α_{y} are calculated for all target sample coordinates (xᵢ, yᵢ). Since the X-Y measurement plane 17 is parallel to the CCD chip 73 and tangent to the corneal apex, each reflection angle αᵢ has an x-component and a y-component given by:
tan(αₓ)=xᵢ/c; tan(α_{y})=yᵢ/c,
where xᵢ and yᵢ are the distance values in the x-direction and the y-direction, respectively, from the measurement axis 19 where the light spots impinge the CCD-chip 73. At step 520 the x- and y-components of the slope angles β for all target sample coordinates (xᵢ, yᵢ) are calculated. Based on the geometrical parameters shown in FIGS. 5 and 6, initial target surface slope angles are determined by:

$${β}^{′} ( x ) = 0.5 \cdot ( arctan ( \frac{h - x}{a + z + Δ} ) - α ( x ) ) ;$$

$${β}^{′} ( y ) = 0.5 \cdot ( arctan ( \frac{h - y}{a + z + Δ} ) - α ( y ) ) ,$$

where x=(b+z+Δ)·tan(α(x))≈(b+z)·tan(α(x)) and y=(b+z+Δ)·tan(α(y))≈(b+z)·tan(α(y)); Δ=r−\sqrt{r²−x²−y²}. At step 525 the slope of the corneal surface T′ at each sample coordinate (xᵢ, yᵢ) is determined by the values tan β′(x) and tan β′(y). The slopes of the reference surface T are calculated at step 530 by the values tan β(x) and tan β(y) as follows:

$$tan ( β ( x ) ) = \frac{x}{\sqrt{{r}^{2} - {x}^{2} - {y}^{2}}} ; tan ( β ( y ) ) = \frac{y}{\sqrt{{r}^{2} - {x}^{2} - {y}^{2}}} .$$

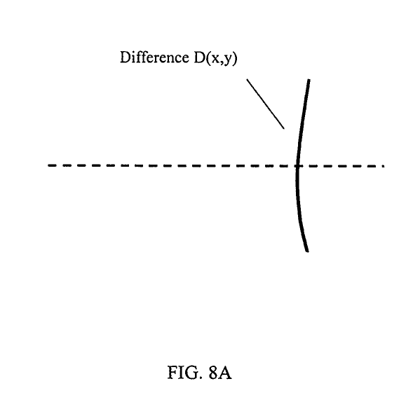

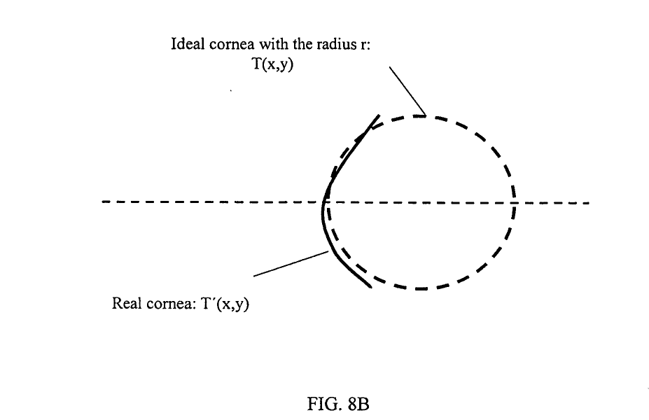

As illustrated in FIGS. 8A and 8B, the difference D(x,y) between the topography (curvatures) of the target and reference surfaces, while not directly measurable, can be expressed at step 535 as the difference in the first derivatives of D(x,y) by:
∂D(x,y)/∂x_{|xi,yi}=tan(β)−tan(β′),
which provides an estimation of the curvature. At step 540 the Zernike coefficients are calculated for all values of D(x,y) to obtain a continuous mapping of the curvature of the corneal surface. The calculation is done similarly to the calculation of the wave front in a Hartmann-Shack wave front sensor system; i.e., the values ∂D(x,y)/∂x,∂y are fitted with the sum of the first derivatives of the Zernike polynomials for all target surface sample points to obtain the Zernike coefficients Cᵢ. With the coefficients, D(x,y) can be calculated at the sample point coordinates as follows:

$$D ( x , y ) = {\sum }_{i} {C}_{i} \cdot {Z}_{i} ( x , y )$$

where Zᵢ(x,y) is the i^{th} Zernike polynomial.

Once the Zernike polynomials are determined, the deviation Δ along the Z-axis from a reference surface to the target surface at each coordinate (xᵢ, yᵢ) can be described as shown at step 545. Thus the value Δ represents a relative topographical elevation value at each sample point.

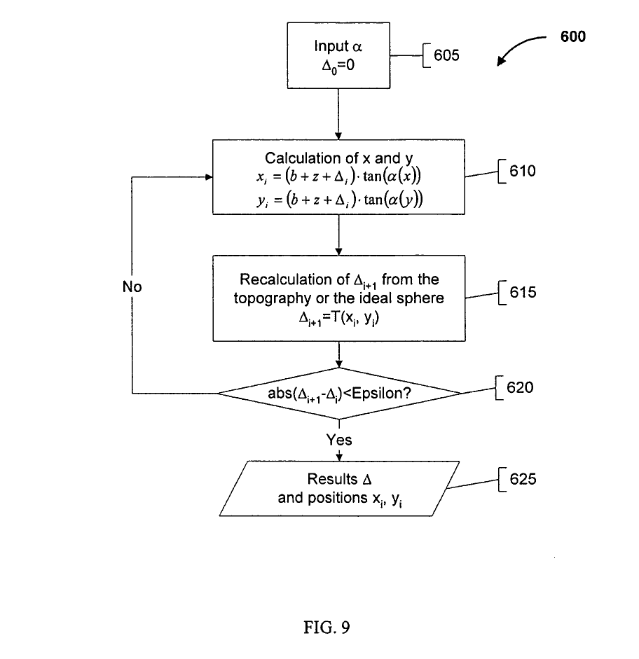

At step 550 the process beginning at step 520 can be iteratively performed for slightly changed values of Δ; i.e., Δ is calculated by an iterative process either from the spherical reference or from the previously calculated topography (prior iteration). FIG. 9 is a flow chart 600 setting forth the iterative process steps stemming from step 545 of FIG. 9. At step 605 the initial value for reflection angle α is input into process 500 for Δ₀=0. At step 610 xᵢ and yᵢ values are calculated as shown for a subsequent value Δᵢ. At step 615 the iterative value Δᵢ₊₁ is determined from the topography determination of T′ at step 545 or from the spherical reference surface T. At step 620 a determination is made whether the absolute difference value [abs(Δᵢ₊₁−Δᵢ)] is less than a predetermined value ε. If the difference value is less than ε, then no further iteration is necessary. If not, steps 610 through 620 are repeated until the difference value is less than ε. According to an illustrative aspect, the change in Δ will be less than 1 mm, therefore the change in the values for (a+z+Δ) and (b+z+Δ) will be minor (approximately 1%). In this case, suitably accurate topographical measurements are obtained with only one or two iterations.

Once the elevation data are known, all of the common ophthalmic topographical maps can be computed; e.g., elevation maps, dioptric power maps, curvature maps, etc. These maps can also be displayed on a video screen or other suitable display medium.

In an illustrative method embodiment, topographical parameter measurements could be made in an ‘online analysis’ manner; i.e., the detection, measurement and display of the topographical parameter information could be obtained substantially simultaneously at a rate up to approximately 50 Hz using a 1.6 GHz Pentium® (Intel) or equivalent processor and, more particularly, at a rate on the order of 25 Hz using an 800 MHz processor. Exemplary online methods and algorithms utilized for wavefront analysis are disclosed in commonly assigned International Application No. PCT/EP2004/008205 filed 22 Jul. 2004, the disclosure of which is hereby incorporated by reference in its entirety to the fullest extent allowed by applicable laws and rules. In an illustrative aspect, 25 images of 500 to 7500 illumination points could be obtained within one second at an online measurement frequency of 25 Hz. The positions of the 500 to 7500 illuminator image centroids imaged by the CCD detector/camera 14 could be located and sorted in approximately 5 ms. This range of illuminators provides a similar number of measurement points as found in current commercially available diagnostic topography devices. Zernike coefficients could then be calculated and topographical parameter (e.g., elevation) information could be determined and displayed in approximately 13 ms or less (utilizing an 800 MHz processor).

According to a related aspect, the video camera 14 could be used to simultaneously obtain iris images. These images could be traced to provide eye movement data over the duration of the online measurement.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description but rather by the claims appended hereto.

We claim:

1. A method for measuring a topographical parameter of a target surface, comprising:
obtaining the positional coordinates of an image of a known array of illumination sources reflected from a reference surface at a known measurement location;
obtaining the corresponding positional coordinates of an image of the known array of illumination sources reflected from a target surface at the known measurement location; and
using a Hartmann-Shack wavefront reconstruction procedure to determine a polynomial-based topographical representation of the target surface.

2. The method of claim 1, further comprising making an online analysis of the topographical parameter at a frequency up to and including 50 Hz.

3. The method of claim 2, comprising making the online measurement at a frequency on the order of 25 Hz over a measurement duration up to about 10 seconds.

4. The method of claim 1, further comprising simultaneously obtaining a plurality of iris or pupil images.

5. The method of claim 4, comprising determining a movement data of the subject's eye based upon at least some of the plurality of iris or pupil images.

6. The method of claim 1, wherein the step of using a Hartmann-Shack wave front reconstruction procedure includes determining a Zernike polynomial representation of the target surface based upon slope data of the reference surface determined from the positional coordinates and slope data of the target surface determined from the corresponding positional coordinates.

7. The method of claim 1, wherein the reference surface is a spherical surface.

8. A method for measuring a topographical parameter of a target surface, comprising:
a) projecting light from a known plurality of light emitting sources onto a surface of the target;
b) imaging a plurality of the projected light sources on the target surface onto a detector;
c) determining a positional coordinate of each imaged light source on the detector, wherein each positional coordinate is determinative of a slope value of the target surface at each respective projected light source coordinate;
d) determining a positional coordinate of each of a corresponding reference surface light source image on the detector, wherein each positional coordinate is determinative of a slope value of the reference surface at each respective projected light source coordinate;
e) determining a difference between the slope of the target surface at each respective projected light source coordinate and the slope of the reference surface at each respective projected light source coordinate, wherein the slope difference represents a change in the deviation of the slopes of the target surface from the slopes of the reference surface; and
f) determining the coefficients of a polynomial for the slope deviation values, wherein a continuous mapping of the target surface is provided by the polynomial representation of the surface.

9. The method of claim 8, further comprising determining a relative elevational deviation value of the target surface at any surface coordinate location based upon the polynomial representation of the surface.

10. The method of claim 9, further comprising iteratively performing steps (c), (e) and (f) based on a previously determined deviation value Δᵢ, until an absolute difference value between Δᵢ₊₁ and Δᵢ is less than a predetermined value.

11. The method of claim 8, wherein the polynomial is a Zernike polynomial.

12. The method of claim 8, wherein the polynomial is at least one of a Taylor series, a Fourier series, a Seidel series, a bicubic spline and an orthogonal two-dimensional function.

13. The method of claim 8, wherein the target surface is an anterior corneal surface.

14. The method of claim 8, comprising constructing a topographical map of the target surface.

15. The method of claim 14, comprising constructing a curvature map.

16. The method of claim 15, further comprising displaying the curvature map on a display medium.

17. The method of claim 9, comprising constructing an elevation map of the target surface.

18. The method of claim 17, further comprising displaying the elevation map on a display medium.

19. The method of claim 8, wherein steps (c) and (d) comprise calculating a centroid location of each of the light source images.

20. The method of claim 8, wherein steps (c) and (d) further comprise calculating the directional components of a reflection angle (α_{x,y}) and a slope angle (β_{x,y}) of at least one of the reference surface and the target surface relative to an X-Y plane that is normal to an axial measurement axis Z.

21. The method of claim 8, further comprising making an online analysis of the topographical parameter at a frequency up to and including 50 Hz.

22. The method of claim 21, comprising making the online measurement at a frequency on the order of 25 Hz over a measurement duration up to about 10 seconds.

23. The method of claim 8, further comprising simultaneously obtaining a plurality of iris or pupil images.

24. The method of claim 23, comprising determining a movement data of the subject's eye based upon at least some of the plurality of iris or pupil images.

25. A topographical parameter measuring device, comprising:
a measurement surface illuminator including a known array of illumination components arranged in a plane that is perpendicular to an axial measurement axis of the device;
a distance measuring component;
a camera and associated detector located along the axial measurement axis of the device in cooperative engagement with the illuminator and the distance measuring component; and
a computational component programmed to calculate reference surface and target surface slope data from reflected reference and target surface illuminator image data and implement a Hartmann-Shack wavefront reconstruction algorithm to determine a polynomial-based topographical representation of the target surface.

26. The device of claim 25, wherein the Hartmann-Shack wavefront reconstruction algorithm uses a Zernike polynomial representation of the wavefront reconstruction.

27. The device of claim 25, wherein the known array of illumination components of the illuminator consists of a plurality of LEDs in a defined pattern having known positions with respect to the axial measurement axis.

28. The device of claim 27, wherein the defined pattern is rotationally symmetric.

29. The device of claim 27, wherein the defined pattern is a plurality of straight lines.

30. The device of claim 27, wherein the defined pattern is a plurality of concentric circular patterns.

31. The device of claim 27, comprising between 30 and 7500 LEDs.

32. The device of claim 31, comprising between 500 and 7500 LEDs.

33. The device of claim 27, comprising between 30 and 300 LEDs.

34. The device of claim 27, wherein the plurality of LEDs emit at least two different colors of light, further wherein the camera is a color sensitive camera.

35. The device of claim 25, further comprising an illuminator controller that provides selective control of the array of illumination components.

36. The device of claim 27, wherein each of the plurality of LEDs emits a main illumination beam component, wherein at least some of the LEDs are oriented to emit their main illumination beam components within a restricted angle range to meet an imposed reflection condition.

37. The device of claim 25, wherein the distance measuring component has a measuring accuracy equal to or better than 0.2 mm with respect to the distance between the surface illuminator and a surface measurement plane of the device.

38. The device of claim 37, wherein the distance measuring component has a measuring accuracy equal to or better than 0.1 mm.

39. The device of claim 25, wherein the distance measuring component is a laser triangulation device.

40. The device of claim 25, wherein the distance measuring component is a slit lamp.

41. The device of claim 25, wherein the distance measuring component is an optical coherence tomography (OCT) device.

42. The device of claim 25, wherein the distance measuring component is an ultrasound device.
